(19) United States
(12) Patent Application Publication
(10) Pub. No.: US 2009/0156565 A1
(43) Pub. Date: Jun. 18, 2009

(54) COMPOSITION AND USE THEREOF IN ENHANCING A THERAPEUTIC EFFECT OF AN ANTIEPILEPTIC DRUG

(75) Inventors: Yogendrasinh H. Raol, Wallingford, PA (US); Amy R. Brooks-Kayal, Media, PA (US)

Correspondence Address: RATNERPRESTIA, P.O. BOX 980, VALLEY FORGE, PA, 19482, US

(73) Assignee: THE CHILDREN'S HOSPITAL OF PHILADELPHIA, Philadelphia, PA (US)

(21) Appl. No.: 11/720,689

(22) PCT Filed: Dec. 5, 2005
(86) PCT No.: PCT/US2005/043805

§ 371 Date: Dec. 22, 2008

Related U.S. Application Data
(60) Provisional application No. 60/633,193, filed on Dec. 3, 2004.

(51) Int. Cl. A61K 31/567; A61K 31/5513; A61P 25/08

(52) U.S. Cl. 514/171; 514/221

(57) ABSTRACT

A composition and a method for the use of the composition for enhancing a therapeutic effect of an antiepileptic drug in treating a seizure in a status epilepticus in an animal. A composition includes a glucocorticoid receptor antagonist and an antiepileptic drug, wherein the glucocorticoid receptor antagonist is present in an amount effective to enhance a therapeutic effect of the antiepileptic drug in treating a seizure in a status-epilepticus in an animal. A method of use of a glucocorticoid receptor antagonist for enhancing a therapeutic effect of an antiepileptic drug in treating a seizure in a status epilepticus in an animal, the method includes administering the glucocorticoid receptor antagonist and the antiepileptic drug, wherein the glucocorticoid receptor antagonist is administered to the animal prior to, contemporaneous with, or subsequent to administering the antiepileptic drug in an amount effective to enhance the therapeutic effect of the antiepileptic drug.

### CROSS-REFERENCE TO RELATED APPLICATIONS

This application claims the benefit of provisional U.S. Patent Application Ser. No. 60/633,193, filed on Dec. 3, 2004, which is incorporated herein in its entirety.

### STATEMENT REGARDING FEDERALLY SPONSORED RESEARCH OR DEVELOPMENT

This research was supported in part by U.S. Government funds (Epilepsy Foundation of America and National Institutes of Health Grant Number NIH NS38595), and the U.S. Government may therefore have certain rights in the invention.

### SPECIFICATION

### Background of the Invention

1. Field of Invention

This invention relates to the use of a glucocorticoid receptor antagonist to increase the efficacy of an antiepileptic drug. Specifically, this invention discloses the use of mifepristone to enhance the efficacy of a benzodiazepine in the treatment of status-epilepticus. In a preferred embodiment, the benzodiazepine is diazepam.

2. Description of Related Art

Epilepsy is a common disorder which has many causes, and it can be very difficult to control clinically, often requiring treatment for many years to keep seizures under control. Researchers have stated that there are no adequate treatments for patients with epilepsy given how many people are affected by this illness (Dichter et al., Drug Therapy 334:1583 (1996)). Due to the widespread availability of reasonably predictive and experimentally accessible animal models of convulsant states, a number of clinically useful anticonvulsants have been prepared and developed. For example, see Cereghino et al., “Introduction,” in ANTIEPILEPTIC DRUGS, 4th ed., pages 1-11 (Rave Press 1995), which states: “In many patients, seizures can be controlled with currently available antiepileptic drugs, but 25 to 30 percent of patients continue to have seizures despite optimal therapy, while many others experience unacceptable side effects.”

The present invention can be used to provide an improved treatment for convulsive disorders such as epilepsy. Specifically, the compositions and methods of the invention can be used to treat status-epilepticus which is evidenced by a reduction of the severity, or duration of seizures, or a complete cessation of a seizure in animal models. Status-epilepticus is characterized by prolonged, self-sustaining seizures lasting about 30 minutes or longer, during which patients do not gain consciousness. Initial treatment of status-epilepticus consists of benzodiazepines such as diazepam. It is well established clinically that the sensitivity of diazepam decreases as the duration of status-epilepticus increases.

The benzodiazepines, a unique class of anxiolytic, anticonvulsant and sedative-hypnotic drugs, are the most widely prescribed “minor tranquilizers” in use today. When administered via the oral route, they become widely distributed throughout the body, particularly in lipid-rich tissues such as adipose and brain.

This invention provides a novel method of utilizing a glucocorticoid receptor antagonist (e.g., mifepristone) as an adjunctive treatment of epilepsy in order to improve the treatment and outcome of status-epilepticus by enhancing the efficacy of an antiepileptic drug (e.g., benzodiazepines). Mifepristone has been noted as being effective at abrogating some of the age-associated electrophysiological changes in the rat hippocampus (Talmi et al., Neurobiol. of Aging 17:9-14, 1996) and also as providing protection against oxidative stress-induced neuronal cell death in the mouse hippocampus (Behl et al., European J. of Neurosci. 9:912-920, 1997).

U.S. Patent Application Publication No. 2002/0065259A1 to Shatzberg et al. pertains to glucocorticoid blocking agents for increasing blood-brain barrier permeability. The reference describes that administration of glucocorticoid blocking agents, including glucocorticoid receptor antagonists concomitant with administration of drugs for treating diseases of the central nervous system (CNS) increases delivery of such drugs into the CNS. Mifepristone is described among glucocorticoid blocking agents.

U.S. Pat. No. 6,620,802 to Schatzberg et al. and U.S. Patent Application Publication No. 2004/0019028 to Schatzberg et al. pertain to the discovery that agents which inhibit the binding of cortisol to its receptors can be used in methods for treating mild cognitive impairment.

U.S. Pat. No. 6,150,349 to Schatzberg et al. pertains to a method for treating psychosis whose pathogenesis is related to glucocorticoid regulatory dysfunction.

U.S. Pat. Nos. 6,380,223 and 6,699,893 to Dow et al. disclose non-steroidal compounds which are selective modulators (i.e., agonists and antagonists) of a steroid receptor, specifically, the glucocorticoid receptor. It also provides pharmaceutical compositions containing these compounds and methods for using these compounds to treat animals requiring glucocorticoid receptor agonist or antagonist therapy.

U.S. Pat. No. 6,645,990 to Askew et al. discloses selected novel urea compounds that are effective for prophylaxis and treatment of diseases, such as epilepsy. The invention encompasses novel compounds, analogs, pro-drugs and pharmaceutically acceptable salts thereof, pharmaceutical compositions and methods for prophylaxis and treatment of diseases and other maladies or conditions involving epilepsy.

U.S. Pat. No. 4,046,890 to Askew et al. discloses a pharmacologically active group of benzodiazepine derivatives said to exhibit, among other things, anticonvulsant, sedative and muscle relaxant activity.

Several patents and patent applications disclose various methods for enhancing the efficacy of therapeutic agents. PCT Application WO 00/54766 to Cook discloses compositions and methods for promoting production gains in animals and for enhancing the efficacy of therapeutic agents. The claims are directed to compositions comprising at least one therapeutic agent and at least one antistress agent and to methods for enhancing the efficacy of a therapeutic agent comprising the co-administration of at least one therapeutic agent and at least one antistress agent to an animal. Mifepristone and diazepam are included in the disclosed antistress agents.

U.S. Pat. No. 4,320,124 to Koe discloses compositions and methods for enhancing binding of a benzodiazepine to central benzodiazepine receptors using phenanthridine type analgesic agents.

U.S. Patent Application Publication No. 2004/0038236 to Wallace et al. discloses methods for diagnosing epilepsy utilizing DNA molecules with mutations disrupting the functioning of an assembled GABAA receptor.

U.S. Pat. No. 4,863,952 to Abe et al. discloses methods of promoting percutaneous drugs (e.g., antiepileptic drugs) adsorption using PCA esters.

U.S. Pat. No. 6,495,120 to McCoy et al. discloses enhancing oral adsorption and bioavailability of pharmaceutical agents (e.g., benzodiazepines) using adsorption enhancers effective for preparing the mucosa to adsorb the pharmaceutical agents. Examples of such enhancers are cyclodextrins and surfactants such as Tween.

Borowicz et al. (Influence of sexual hormone antagonists on the anticonvulsant action of conventional antiepileptic drugs against electrically- and pentylenetetrazol-induced seizures in mice. European Neuropsychopharmacology 14 (2004); pp. 77-85) describe a study of three gonadal steroid antihormones, tamoxifen (TXF, an estrogen antagonist), cyproterone acetate (CYP, an antiandrogen) and mifepristone (MIF, a progesterone antagonist) alone or in combination with conventional antiepileptics in a study of electrically- and pentylenetetrazol-induced seizures in mice. The authors determined that mifepristone when used alone had no effect on electrically-induced seizures in mice. Further, mifepristone when used in combination with tested antiepileptic drug including clonazepam, carbamazepine, and phenobarbital failed to affect their protective action against maximal electricroshock-induced seizures in mice (p. 79, Table 1). Diazepam was not one of the antiepileptic drugs utilized in this study. The authors also demonstrated that the protective activity of clonazepam against maximum electroshock was improved by co-administration of TXF in female mice and the co-administration of CYP in male mice. The authors also determined that all three antihormones including mifepristone when used in the combination with tested antiepileptic drug including clonazepam, carbamazepine, and phenobarbital failed to affect their protective action against the pentylenetetrazol-induced seizure in mice.

Benzodiazepines depress consciousness and respiratory drive in a dose-dependent manner (Lowenstein D H, Alldredge B K. (1998) Status epilepticus. N Engl J. Med. 338(14):970-6). Thus, the amount of benzodiazepines that can be administered in the initial treatment of status epilepticus is limited. The clinical impact of this limitation is that for many patients smaller doses of benzodiazepines must be repeatedly administered to achieve complete control of seizure activity. The time delay involved in repeated administration is often associated with a progressive decrease in benzodiazepine efficacy, as experimental and clinical observations show that status epilepticus of longer duration is less responsive to drug therapy than that of shorter duration (Lowenstein D H, Alldredge B K. Status epilepticus at an urban public hospital in the 1980s. Neurology 1993; 43:483-8).

Despite the foregoing developments, it is desired to provide an efficient method of increasing the efficacy of an antiepileptic drug, for example a benzodiazepine, through the use of a glucocorticoid receptor antagonist, for example, mifepristone, to treat status-epilepticus.

These and other advantages of the present invention, as well as additional inventive features, will be apparent from the description of the invention provided herein.

All references cited herein are incorporated by reference in their entireties.

### BRIEF SUMMARY OF THE INVENTION

Accordingly, the invention provides a method of use of a glucocorticoid receptor antagonist for enhancing a therapeutic effect of an antiepileptic drug in treating a seizure in a status epilepticus in an animal, the method includes administering the glucocorticoid receptor antagonist and the antiepileptic drug, wherein the glucocorticoid receptor antagonist is administered to the animal prior to, contemporaneous with, or subsequent to administering the antiepileptic drug in an amount effective to enhance the therapeutic effect of the antiepileptic drug.

In certain embodiments of the method of the invention, the glucocorticoid receptor antagonist is administered to the animal prior to, contemporaneous with, or subsequent to a start of the seizure. In certain embodiments, the glucocorticoid receptor antagonist is administered to the animal prior, contemporaneous with or subsequent to beginning of the seizure.

In certain embodiments of the method of the invention, the glucocorticoid receptor antagonist is mifepristone. In certain embodiments, the benzodiazepine is diazepam, lorazepam, or midazolam. In a preferred embodiment, the benzodiazepine is diazepam. In one embodiment, the benzodiazepine is administered intravenously at a dose of about 0.1 to about 10 mg/kg. In another embodiment, the dose of the benzodiazepine is 0.1 to 0.2 mg/kg. In a further embodiment, the mifepristone is administered orally at a dose of about 10 to about 800 mg. In a preferred embodiment, the dose of the mifepristone is 600 mg. In another embodiment, the mifepristone is administered orally and the antiepileptic drug is administered intravenously.

In another embodiment, the therapeutic effect is a reduction in length or severity of a seizure, or a complete cessation of a seizure. In a further embodiment, the enhancing is due to an inhibition or blocking of a glucocorticoid receptor or a progesterone receptor.

In another aspect, the invention provides a composition comprising a glucocorticoid receptor antagonist and an antiepileptic drug, wherein the glucocorticoid receptor antagonist is present in an amount effective to enhance a therapeutic effect of the antiepileptic drug in treating a seizure in a status-epilepticus in an animal. Inventors have discovered that administration of a glucocorticoid receptor antagonist before, during and after the onset of the seizure reduces the progressive resistance to antiepileptic drugs associated with the prolonged seizure activity.

In certain embodiments of the composition of the invention, the glucocorticoid receptor antagonist is mifepristone. In certain embodiments, the antiepileptic drug is a benzodiazepine.

In certain embodiments, the benzodiazepine is diazepam, lorazepam, or midazolam. In a preferred embodiment, the benzodiazepine is diazepam. In certain embodiments, the benzodiazepine is administered at a dose of about 0.1 to about 10 mg/kg. In a preferred embodiment, the dose of the benzodiazepine is 0.1 to 0.2 mg/kg. In another embodiment, the mifepristone is administered at a dose of about 10 to about 800 mg. In a preferred embodiment, the dose of the mifepristone is 600 mg. In certain embodiments, the therapeutic effect comprises a reduction in length or severity of a seizure and a complete cessation of a seizure.

In another embodiment, the composition further comprises a pharmaceutical carrier. In a preferred embodiment, the pharmaceutical carrier is selected from the group consisting of water, a Ringer's solution, dextrose solution, 5% human serum albumin and a liposome. In another embodiment, the composition further comprises an additional enhancer to enhance the therapeutic effect.

In another aspect, the invention provides a kit for enhancing a therapeutic effect of an antiepileptic drug in treating a seizure in a status epilepticus in an animal, the kit comprises mifepristone, an antiepileptic drug and a pharmaceutical carrier.

### BRIEF DESCRIPTION OF SEVERAL VIEWS OF THE DRAWINGS

The invention will be described in conjunction with the following drawings in which like reference numerals designate like elements and wherein:

FIG. 1 is a histogram representing the ability of mifepristone treatment to increase the efficacy of repeated diazepam doses (mg/kg body weight) to terminate seizures in rats. It was observed that the cumulative diazepam dose required to stop seizures was lower in mifepristone treated rats.

FIG. 2 is a histogram representing the ability of mifepristone treatment to increase the efficacy of a single dose of diazepam in terminating seizures in rats.

FIG. 3A is an electroencephalogram (EEG) recorded from cortex (Cx) and hippocampus (Hp) showing that rats that received mifepristone had a lower frequency of spike-wave discharges and seizure recurrence 24 hours after pilocarpine injection.

FIG. 3B is an EEG showing that rats that did not receive mifepristone had a higher frequency of spike-wave discharges and seizure recurrence 24 hours after pilocarpine injection.

FIG. 4 is a bar graph comparing the severity of on seizures of rats depending on treatment with mifepristone treatment after the onset of a seizure.

### DETAILED DESCRIPTION OF THE INVENTION

The present invention is based upon a discovery by inventors that a glucocorticoid receptor antagonist can be utilized to increase the efficacy of an antiepileptic drug to treat a seizure in a status-epilepticus. The invention provides a composition comprising a glucocorticoid receptor antagonist and an antiepileptic drug, wherein the glucocorticoid receptor antagonist is present in an amount effective to enhance a therapeutic effect of the antiepileptic drug in treating a seizure in a status-epilepticus in an animal. A non-limiting example of the glucocorticoid receptor antagonist is mifepristone. A non-limiting example of the antiepileptic drug is a benzodiazepine. In certain embodiments, the benzodiazepine is diazepam, lorazepam, or midazolam. Preferred benzodiazepine is diazepam. In certain embodiments, the dose of the benzodiazepine is about 0.1 to about 10 mg/kg. In a preferred embodiment, the dose of the benzodiazepine is 0.1 to 0.2 mg/kg. In another embodiment, the mifepristone is administered at a dose of about 10 to about 800 mg. In a preferred embodiment, the dose of the mifepristone is 600 mg. In certain embodiments, the therapeutic effect comprises a reduction in length or severity of a seizure and a complete cessation of a seizure.

In another embodiment, the composition further comprises a pharmaceutical carrier. In a preferred embodiment, the pharmaceutical carrier is selected from the group consisting of water, a Ringer's solution, dextrose solution, 5% human serum albumin and a liposome. In another embodiment, the composition further comprises an additional enhancer to enhance the therapeutic effect.

### DEFINITIONS

As used herein, each of the following terms has the meaning associated with it in this section, absent an express indication to the contrary.

The articles “a” and “an” are used herein to refer to one or to more than one (i.e. to at least one) of the grammatical object of the article. By way of example, “an enhancer” means one v or more than one enhancer.

The term “glucocorticoid receptor” refers to a family of intracellular receptors, also referred to as the cortisol receptor, which specifically binds to cortisol and/or cortisol analogs.

The term includes isoforms of glucocorticoid receptor, recombinant glucocorticoid receptor and mutated glucocorticoid receptor.

The term “mifepristone” refers to a family of compositions also referred to as RU486, or RU38.486, or 17-beta-hydroxy-11-beta-(4-dimethyl-aminophenyl)-17-alpha(1-propynyl)-estra-4,9-dien-3-one), or 11-beta-(4-dimethylaminophenyl)-17-beta-hydroxy-17-alpha-(1-propynyl)-estra-4,9-dien-3-one), or analogs thereof, which bind to the glucocorticoid receptor, typically with high affinity, and inhibit the biological effects initiated/mediated by the binding of any cortisol or cortisol analogue to a glucocorticoid receptor. Chemical names for mifepristone vary; for example, mifepristone has also been termed: 11B-[p-(Dimethylamino)phenyl]-17β-hydroxy-17-(1-propynyl)-estra-4,9-dien-3-one; 11B-(4-dimethyl-aminophenyl)-17β-hydroxy-17A-(prop-1-ynyl)-estra-4,9-dien-3-one; 17B-hydroxy-11B-(4-dimethylaminophenyl-1)-17A-(propynyl-1)-estra-4,9-diene-3-one; 17B-hydroxy-11B-(4-dimethylaminophenyl-1)-17A-(propynyl-1)-E; (11B, 17B)-11-[4-dimethylamino)phenyl]-17-hydroxy-17-(1-propynyl)estra-4,9-dien-3-one; and 11B-[4-(N,N-dimethylamino)phenyl]-17A-(prop-1-ynyl)-D-4,9-estradiene-17B-ol-3-one. Mifepristone is a yellow powdery substance and is available commercially (Sigma, St. Louis, Mo.).

The term “composition” refers to a combination of at least two compounds in an animal, for example, mifepristone and diazepam, wherein the compounds may be administered concurrently or sequentially with respect to one another to the animal. That is, a first compound (i.e., mifepristone) may be administered prior to, simultaneous with or subsequent to a second compound (i.e., an antiepileptic drug). The compounds coexist within the animal following administration for at least a minimum amount of time allowing enhancing a therapeutic effect of said antiepileptic drug in treating status-epilepticus. The term “composition” also contemplates a combination of at least two compounds in a container prior to administering it to an animal.

In one aspect, the invention includes a composition comprising mifepristone and an antiepileptic drug, wherein said mifepristone is present in an amount effective to enhance a therapeutic effect of said antiepileptic drug in treating status-epilepticus.

The term “antiepileptic drug” refers to a compound that is used to alleviate a symptom of epilepsy, specifically a seizure. These antiepileptic drugs are typically categorized according to their mechanism of action and include sodium channel blockers, calcium current inhibitors, gamma-aminobutyric acid (GABA) enhancers, glutamate blockers, carbonic anhydrase inhibitors, hormones, and drugs with unknown mechanisms of action. In one embodiment, the antiepileptic drug is selected from the group consisting of a benzodiazepine, primidone, gabapentin, lamotrigine, felbamate, topiramate and fosphenyloin phenyloin, carbamazepine, valproic acid and phenobarbital. In a preferred embodiment, the antiepileptic drug is a benzodiazepine. In another embodiment, the benzodiazepine is selected from the group consisting of diazepam, lorazepam, clonazepam, clobazam, midazolam, temazepam, loprazolam, lormetazepam alprazolam, bromazepam, flunitrazepam, nitrazepam, oxazepam, triazolam. In a preferred embodiment, the benzodiazepine is diazepam. The antiepileptic drugs are sold by various pharmaceutical companies and may be purchased by a licensed physician or medical facility.

The term “status-epilepticus” describes an epileptic seizure that lasts more than 15-30 minutes or a constant or near-constant state of having seizures. In certain situations, epileptic seizure that are shorter, such as, for example, 5 minutes of continuous generalized convulsive activity may require intravenous antiepileptic drug therapy.

In another aspect, the invention provides a method of use of composition of the invention for enhancing a therapeutic effect of an antiepileptic drug in treating a seizure in a status epilepticus in an animal, the method comprising administering the glucocorticoid receptor antagonist and administering the antiepileptic drug, wherein the glucocorticoid receptor antagonist is administered in an amount effective to enhance the therapeutic effect of the antiepileptic drug in treating the seizure in the status-epilepticus in the animal. The glucocorticoid receptor antagonist can be administered to the animal prior to, contemporaneous with, or subsequent to administering the antiepileptic drug. In certain embodiments of the method, the glucocorticoid receptor antagonist is mifepristone.

In certain embodiments of the method of the invention, the glucocorticoid receptor antagonist is administered to the animal prior to, contemporaneous with, or subsequent to a start of the seizure. Example 1 demonstrates the effect of administering mifepristone prior to the start of the seizure. Example 2 demonstrates the effect of administering mifepristone after onset of the seizure.

There is substantial clinical and experimental evidence that timing of anti-epileptic drug administration relative to the onset of status epilepticus plays an important role in determining of how effective the drug is in stopping the seizure. Experimental and clinical observations show that status epilepticus of a longer duration is less responsive to drug therapy than that of a shorter duration (Lowenstein D H, Alldredge B K. Status epilepticus at an urban public hospital in the 1980s. Neurology 1993; 43:483-8). For example, it was found that seizures were stopped by firstline therapy (usually diazepam followed by phenyloin) in 80 percent of patients when treatment was begun within 30 minutes of the onset of the seizures (Lowenstein D H, Alldredge B K. Status epilepticus at an urban public hospital in the 1980s. Neurology 1993; 43:483-8). In contrast, the response rate was less than 40 percent when treatment was begun two hours or more after the onset of the seizures. In rats, status epilepticus becomes progressively less responsive to treatment with diazepam as electrographic seizures continue (Walton N Y, Treiman D M. Response of status epilepticus induced by lithium and pilocarpine to treatment with diazepam. Exp Neurol 1988; 101:267-75). Thus, administering mifepristone after the seizure onset was not expected to be as efficacious as its administration prior to seizure onset. Surprisingly, the inventors have observed that pre-treatment (prior to seizure onset) and treatment up to 15 minutes after status-epilepticus onset were both effective in increasing the efficacy of diazepam in terminating seizures.

In one embodiment, the therapeutic effect is a reduction in the length or severity of a seizure, or a complete cessation of a seizure. In another embodiment, the effect is due to an inhibition or blocking of a glucocorticoid receptor or a progesterone receptor. In a further embodiment, the animal is a mammal. In a preferred embodiment, the mammal is a human, mouse, rat, rabbit, dog, cat cow or horse. In a more preferred embodiment, the mammal is human.

The amount of agents such as an antiepileptic drug and a glucocorticoid receptor antagonist, routes of delivery and timing of administration of the compositions of the invention varies in accordance with the compound employed, the animal species, bodyweight, age and whether the treatment is therapeutic or prophylactic. Accordingly, in most cases dosing and dosages will be carried out according to manufacturer's instructions or as otherwise known in the art.

The benzodiazepine and mifepristone compounds can be administered concurrently as a single dosage composition or formulation. Alternatively, they can be administered concurrently as separate dosage forms. Still further, the mifepristone can be administered either before or after administration of the benzodiazepine. When given separately, mifepristone and benzodiazepine can be given by the same or different routes of administration. It is, however, for convenience of the patient and physician, preferred to use the two components; e.g., mifepristone and benzodiazepine, as a single composition; e.g., a mixture, via a single route of administration.

Single and multiple dosing regimes are contemplated. Multiple dosing regimes may comprise administration of two or more agent doses to different sites on or by different routes of administration to an animal at the same time. In one embodiment, multiple dosing regimes may comprise the administration of two or more doses of agents to an animal over a period of time covering hours, days and weeks. The amount of agents in the composition may vary within a broad range, as long as effectiveness is maintained. In one embodiment, benzodiazepine is administered intravenously to a child at a concentration between 0.1-0.2 mg/kg. In another embodiment, benzodiazepine is administered intravenously to an adult at a concentration between 5-10 mg/kg and rectally at a concentration between 0.2-0.5 mg/kg. Generally, the optimal dose of diazepam administered is at a concentration between 0.1-10 mg/kg. In a further embodiment, mifepristone is administered orally at a concentration between 10-800 mg. In a preferred embodiment, mifepristone is administered orally at a concentration of 600 mg.

Further, more than one type of antiepileptic drug can be administered e.g., benzodiazepine and phenyloin.

The compositions of the invention can be incorporated into pharmaceutical compositions suitable for administration. Such compositions typically comprise the drug(s) and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), intra-oral, transdermal (topical), transmucosal, and rectal administration. In one embodiment, the mifepristone is administered orally and the benzodiazepine is administered intravenously. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., mifepristone or diazepam) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (Mountain View, Calif.) and Nova Pharmaceuticals, Inc (Lake Elsinore, Calif.). Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 to Eppstein et al.

It is especially advantageous to formulate oral or parenteral compositions in a dosage unit form for ease of administration and uniformity of dosage. The dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a kit, container, pack, or dispenser together with instructions for administration. In another aspect, the invention also contemplates a kit for enhancing a therapeutic effect of an antiepileptic drug in treating a seizure in a status epilepticus in an animal, wherein the kit comprises mifepristone, an antiepileptic drug and a pharmaceutical carrier.

The invention will be illustrated in more detail with reference to the following Examples, but it should be understood that the present invention is not deemed to be limited thereto.

### EXAMPLES

### Example 1

### Treatment of Rats with Mifepristone Before Onset of SE Increases Efficiency of Diazepam

Mifepristone is a yellow powdery substance and is available commercially (Sigma, St. Louis, Mo.). Adult male Sprague-Dawley rats were implanted with electrodes in CA1 and frontal cortex. After one week of recovery, rats were put in recording cages and connected to EEG monitoring system for continuous video-EEG recording. Rats were then treated with mifepristone (25 mg/kg body weight) one hour before the pilocarpine injection. Thirty minutes later rats were treated with scopalamine (1 mg/kg body weight) to reduce peripheral side effects of pilocarpine. Rats were injected with pilocarpine (385 mg/kg body weight) 30 minutes after the scopalamine injection to induce status-epilepticus. One hour after declared status-epilepticus, rats were treated with diazepam (6 mg/kg) to stop the seizures. Every two hours rats were monitored for signs of behavioral seizures and if required they were injected with 3 mg/kg diazepam until they completely stop seizing. The process was repeated until the rats completely stopped seizing. Rats that were pretreated with mifepristone required a significantly (unpaired t-test, p<0.0005) lower dose of diazepam (7.5 mg/kg±0.41, n=14) compared to those that did not receive mifepristone (10.2 mg/kg±0.5, n=20). This example was presented by inventors in an article titled “Effects of Glucocorticoid Receptor Activity on Diazepam Efficacy in Terminating Status-Epilepticus in Adult Rats,” Epilepsia Vol. 45, Suppl. 7, page 146, October, 2004.

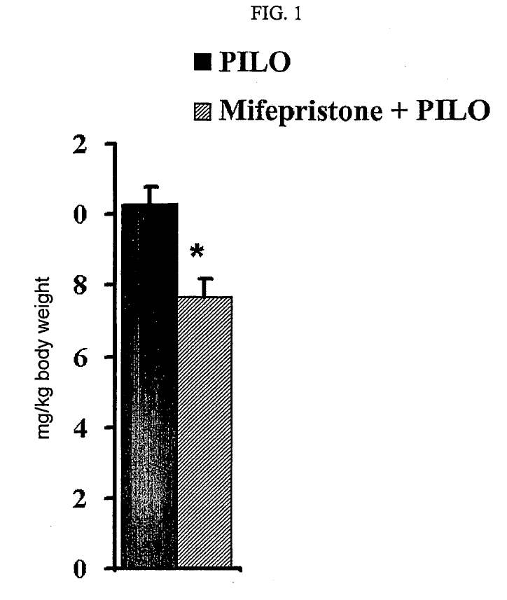

As shown in FIG. 1, mifepristone treatment increases efficacy of repeated diazepam doses to terminate seizures. The histogram represents mean (±SEM) cumulative diazepam dose (mg/kg body weight) required to stop behavioral seizures. Rats that were pretreated with mifepristone (gray bar) before pilocarpine induced status-epilepticus required a 26.5% lower cumulative diazepam dose to terminate behavioral seizures as compared to pilocarpine treated rats that were not pretreated with mifepristone (black bar). *P<0.0005. n=20 for pilo group and n=14 for mifepristone+pilo group.

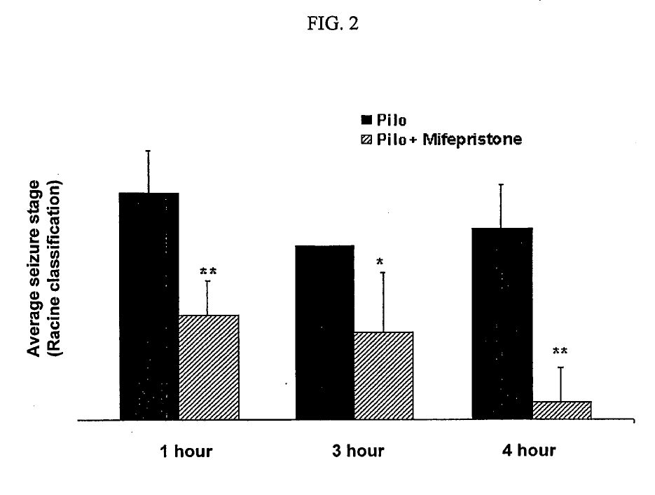

As shown in FIG. 2, mifepristone treatment increases efficacy of a single dose of diazepam in terminating seizures. The histogram represents mean (±SD) seizures stage (based on Racine classification of stage 1-5; 1 being least severe and 5 being most severe) 1, 3 and 4 hours after a single diazepam dose (7.5 mg/kg body weight) given 30 minutes after status-epilepticus. Four rats out of 5 that were pretreated with mifepristone (gray bar) before pilocarpine induce status-epilepticus completely stopped behaviorally seizing after 4 hours of diazepam treatment, whereas, in rats that were not pretreated with mifepristone (black bar) only 1/5 stopped seizing by 4 hours. (*P<0.01; **P<0.001. n=5).

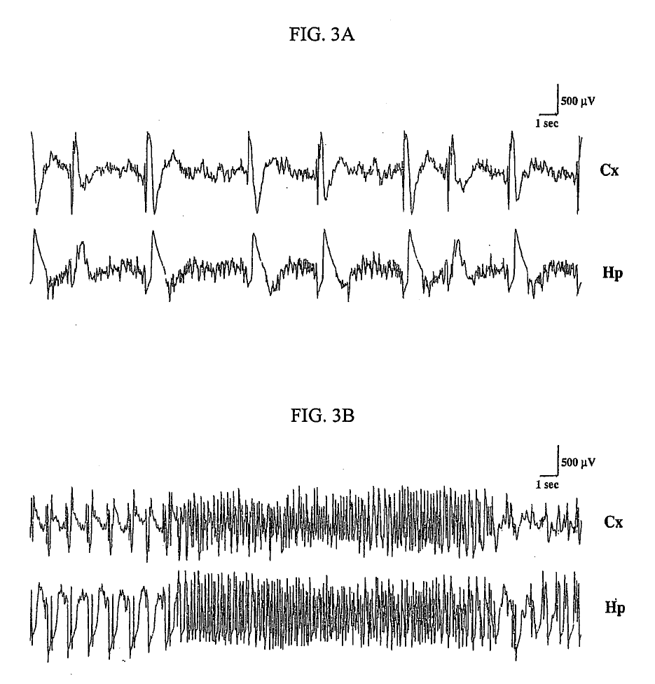

As shown in FIGS. 3A and B, mifepristone treatment improves overall outcome of status-epilepticus over next 24 hours. Rats that received mifepristone (FIG. 3A) had lower frequency of spike-wave discharges and seizure recurrence 24 h hours after pilocarpine injection than rats without treatment (FIG. 3B).

Overall, this study showed that rats pretreated with mifepristone prior to status-epilepticus induction required much lower doses of diazepam to stop seizures. It is well established clinically that as seizures become more prolonged they are more difficult to treat and the efficacy of benzodiazepines diminishes greatly. Thus, the increased efficacy of diazepam induced by mifepristone to block status-epilepticus is very useful clinically.

### Example 2

### Treatment of Rats with Mifepristone After Onset of SE Increases Efficiency of Diazepam

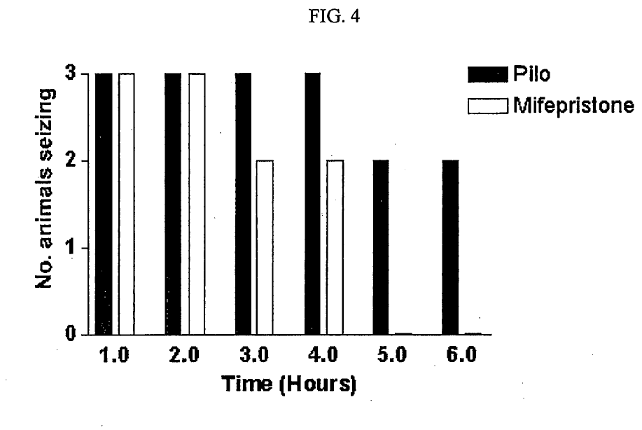

In this experiment, mifepristone was given 15 minutes after onset of SE, but prior to administering the first dose of diazepam (DZ). Diazepam (7.5 mg/kg) was given after 1 hour of onset of SE. After DZ treatment, rats were monitored hourly for behavioral seizures. It was found that at 3 to 6 hours after DZ treatment, significantly more mifepristone treated rats had stopped seizing compared to rats that did not receive mifepristone treatment. Notably, at 5 and 6 hours after DZ treatment, 100% of mifepristone treated rats stopped seizing whereas only 33% of rats not receiving mifepristone stopped seizing (p<0.018, Kaplan-Meier analysis). The results of this experiment are shown in FIG. 4. These results support the clinical utility of mifepristone in the treatment of status epilepticus by demonstrating that mifepristone given after the onset of seizures is effective in enhancing the therapeutic effects of benzodiazepines.

While the invention has been described in detail and with reference to specific examples thereof, it will be apparent to one skilled in the art that various changes and modifications can be made therein without departing from the spirit and scope thereof.

1. A method of use of a glucocorticoid receptor antagonist for enhancing a therapeutic effect of an antiepileptic drug in treating a seizure in a status epilepticus in an animal, the method comprising:
administering the glucocorticoid receptor antagonist; and
administering the antiepileptic drug, wherein the glucocorticoid receptor antagonist is administered in an amount effective to enhance the therapeutic effect of the antiepileptic drug in treating the seizure in the status-epilepticus in the animal, provided that the glucocorticoid receptor antagonist is administered to the animal prior to, contemporaneous with, or subsequent to administering the antiepileptic drug, and thereby enhancing the therapeutic effect of the antiepileptic drug.

2. The method of claim 1, wherein the glucocorticoid receptor antagonist is administered to the animal prior to, contemporaneous with, or subsequent to a start of the seizure.

3. The method of claim 1, wherein the glucocorticoid receptor antagonist is administered to the animal contemporaneous with or subsequent to beginning of the seizure.

4. The method of claim 1, wherein the antiepileptic drug is a benzodiazepine.

5. The method of claim 1, wherein the glucocorticoid receptor antagonist is mifepristone.

6. The method of claim 5, wherein mifepristone is administered orally.

7. The method of claim 5, wherein the antiepileptic drug is administered intravenously.

8. The method of claim 5, wherein mifepristone is administered at a dose of about 10 to about 800 mg.

9. The method of claim 8, wherein the dose of mifepristone is at most 600 mg.

10. (canceled)

11. The method of claim 4, wherein the benzodiazepine is diazepam, lorazepam, or midazolam.

12. The method of claim 4, wherein the benzodiazepine is administered intravenously.

13. The method of claim 4, wherein the benzodiazepine is administered at a dose of about 0.1 to about 10 mg/kg.

14. The method of claim 13, wherein the dose of the benzodiazepine is 0.1 to 0.2 mg/kg.

15. The method of claim 1, wherein the therapeutic effect comprises a reduction in length or severity of a seizure and a complete cessation of a seizure.

16. The method of claim 1, wherein said enhancing is due to an inhibition or blocking of a glucocorticoid receptor.

17. The method of claim 1, wherein said enhancing is due to an inhibition or blocking of a progesterone receptor.

18. A composition comprising a glucocorticoid receptor antagonist and an antiepileptic drug, wherein the glucocorticoid receptor antagonist is present in an amount effective to enhance a therapeutic effect of the antiepileptic drug in treating a seizure in a status-epilepticus in an animal.

19. The composition of claim 18, wherein the glucocorticoid receptor antagonist is mifepristone.

20. The composition of claim 19, wherein the antiepileptic drug is a benzodiazepine.

21. The composition of claim 20, wherein the benzodiazepine is diazepam, lorazepam, or midazolam.

22. The composition of claim 20, wherein the benzodiazepine is present in a dose of about 0.1 to about 10 mg/kg.

23. The composition of claim 22, wherein the dose of the benzodiazepine is 0.1 to 0.2 mg/kg.

24. The composition of claim 19, wherein mifepristone is present in a dose of about 10 to about 800 mg.

25. The composition of claim 24, wherein the dose of mifepristone is at most 600 mg.

26. The composition of claim 18, wherein the therapeutic effect comprises a reduction in length or severity of a seizure and a complete cessation of a seizure.

27. The composition of claim 18 further comprising a pharmaceutical carrier.

28. The composition of claim 27, wherein the pharmaceutical carrier is selected from the group consisting of water, a Ringer's solution, a dextrose solution, human serum albumin and a liposome.

29. A kit for enhancing a therapeutic effect of an antiepileptic drug in treating a seizure in a status epilepticus in an animal, the kit comprises mifepristone, an anti epileptic drug and a pharmaceutical carrier.

30. The kit of claim 29, wherein the antiepileptic drug is a benzodiazepine.

31. The method of claim 1, wherein the animal is a mammal.

32. The method of claim 31, wherein the mammal is a human.
